(19) United States
(12) Patent Application Publication
(10) Pub. No.: US 2017/0364650 A1
(43) Pub. Date: Dec. 21, 2017

(54) MANAGING A THERAPEUTIC STATE BASED ON COGNITIVE, CONTEXTUAL, AND LOCATION-BASED ACTION RECOGNITION

(71) Applicant: International Business Machines Corporation, Armonk, NY (US)

(72) Inventors: Lisa Seacat DELUCA, Baltimore, MD (US); Jeremy Adam GREENBERGER, Raleigh, NC (US); James Robert KOZLOSKI, New Fairfield, CT (US); John Jeremy RICE, Mohegan Lake, NY (US)

(21) Appl. No.: 15/185,120

(22) Filed: Jun. 17, 2016

(51) Int. Cl. G06F 19/00; A61B 5/11; G06F 1/16; G06N 99/00; A61B 5/00; G06N 7/00

(52) U.S. Cl. CPC G06F 19/3418; A61B 5/0077; G06N 7/005; G06F 1/163; G06N 99/005; A61B 5/0022; A61B 5/112; A61B 5/1116; A61B 2562/0219; A61B 2562/0204

(57) ABSTRACT

Disclosed is a novel system, computer program product, and method for managing a therapeutic state of a subject of interest. A combination of location-based information, contextual-based information, and cognitive-based information is accessed for the subject of interest. A machine learning algorithm calculates a therapeutic state of the subject of interest using as inputs the location-based information, contextual-based information, and cognitive-based information. A predefined policy associated with the therapeutic state of the subject of interest. Based on the policy, the vital signs of the subject of interest are monitored.

### BACKGROUND

The present invention generally relates to cloud services, and more particularly to the field of computer systems for managing a therapeutic state of a subject of interest.

Therapeutic drug monitoring (TDM) is the clinical practice of measuring specific drugs at designated intervals to maintain a constant concentration in a patient's bloodstream, thereby optimizing individual dosage regimens. It is unnecessary to employ TDM for the majority of medications, and it is used mainly for monitoring drugs with narrow therapeutic ranges, drugs with marked pharmacokinetic variability, medications for which target concentrations are difficult to monitor, and drugs known to cause therapeutic and adverse effects. The process of TDM is predicated on the assumption that there is a definable relationship between dose and plasma or blood drug concentration, and between concentration and therapeutic effects. TDM begins when the drug is first prescribed, and involves determining an initial dosage regimen appropriate for the clinical condition and such patient characteristics as age, weight, organ function, and concomitant drug therapy. When interpreting concentration measurements, factors that need to be considered include the sampling time in relation to drug dose, dosage history, patient response, and the desired medicinal targets. The goal of TDM is to use appropriate concentrations of difficult-to-manage medications to optimize clinical outcomes in patients in various clinical situations.

BLE Beacons (or Bluetooth low energy beacons) and Wi-Fi sensors are amongst the most popular means of providing enhanced experiences for customers of venues, such as stadiums, airports, retail stores and hospitals. They can be used for a wide range of use cases from sales promotions to building security. These devices transmit a wireless Bluetooth or Wi-Fi signal to enabled devices, such as smart phones, that can notify an application on the device that it is within the proximity of a specified location which could eventually present a promotion or alert.

### SUMMARY

Drug concentration in a subject of interest cannot be routinely measured, but the desired or adverse effects may correlate better with plasma or blood concentrations than they do with dose. For a few drugs, concentration measurements are a valuable surrogate of drug exposure, particularly if there is no simple or sensitive measure of effect.

Many times there is a large inter-individual variation between dose and effect, for example when there is large pharmacokinetic variation, individualizing drug dosage is difficult. This is particularly relevant for drugs with a narrow target range or concentration-dependent pharmacokinetics. Similarly, variations within an individual can occur over time for a range of reasons with some drugs, and therapeutic drug monitoring could then be useful.

Disclosed is a novel system, computer program product, and method for managing a therapeutic state of a subject of interest. A combination of location-based information, contextual-based information, and cognitive-based information is accessed for the subject of interest. A machine learning algorithm calculates a therapeutic state of the subject of interest using as inputs the location-based information, contextual-based information, and cognitive-based information. A predefined policy associated with the therapeutic state of the subject of interest. Based on the policy, the vital signs of the subject of interest are monitored.

### BRIEF DESCRIPTION OF THE DRAWINGS

The accompanying figures wherein reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a block diagram illustrating one example of an operating environment according to one embodiment of the present invention;

FIG. 2 is a diagram illustrating a view of a stateless transaction manager that is part of the machine learning system of FIG. 1;

FIG. 3 is a flow diagram illustrating;

FIG. 4 illustrates one example of a cloud computing node according to one embodiment of the present invention;

FIG. 5 illustrates one example of a cloud computing environment according to one example of the present invention; and

FIG. 6 illustrates abstraction model layers according to one example of the present invention.

### DETAILED DESCRIPTION

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated. The terminology used herein is for the purpose of describing particular embodiments only and is not intended to be limiting of the invention.

The present invention provides a method, system and apparatus for managing a therapeutic state of a subject of interest. The present invention makes use of a combination of location-based information, contextual-based information, and cognitive-based information. A machine learning algorithm calculates a therapeutic state of the subject of interest using as inputs the location-based information, contextual-based information, and cognitive-based information. A predefined policy associated with the therapeutic state of the subject of interest. Based on the policy, the vital signs of the subject of interest are monitored.

### Non-Limiting Definitions

The terms “a”, “an” and “the” are intended to include the plural forms as well, unless the context clearly indicates otherwise.

The terms “comprises” and/or “comprising”, when used in this specification, specify the presence of stated features, steps, operations, elements, and/or components, but do not preclude the presence or addition of one or more other features, integers, steps, operations, elements, components, and/or groups thereof.

The term “cognitive state” is defined as a representation of measures of a subject of interest's total behavior over some period of time (including musculoskeletal gestures, speech gestures, posture, gait, internal physiological changes, measured by imaging devices, microphones, physiological and kinematic sensors in a high dimensional measurement space) within a lower dimensional feature space. In one example certain feature extraction techniques for identifying certain cognitive states. The cognitive state is derived using both historical and real-time physical, psychological, and/or biometric factors. Specifically, the reduction of a set of behavioral measures over some period of time to a set of feature nodes and vectors, corresponding to the behavioral measures' representations in the lower dimensional feature space, is used to identify the emergence of a certain cognitive state over that period of time. The relationship of one feature node to other similar nodes through edges in a graph corresponds to the temporal order of transitions from one set of measures and the feature nodes and vectors to another. Some connected subgraphs of the feature nodes are herein defined as a cognitive state. The present disclosure describes the analysis, categorization, and identification of these cognitive states by means of further feature analysis of subgraphs, including dimensionality reduction of the subgraphs, for example by means of graphical analysis, which extracts topological features and categorizes the resultant subgraph and its associated feature nodes and edges within a subgraph feature space.

The term “Bluetooth Smart Beacons” is used to mean Bluetooth low energy proximity sensing devices that transmit a unique identifier picked up by a compatible app or operating system. The identifier and several bytes sent with it can be used to determine the device's physical location, track customers, or trigger a location-based action on the device such as a check-in on social media or a push notification. Example of Bluetooth low energy proximity sending include iBeacon and Eddystone. These can include nearable and wearable technologies as well.

A “Hauser diary” is a record of a drug's effectiveness for a specified disease. This is widely used in with Parkinson's disease to explore implications for practical use in clinical trials. For Parkinson, the diary includes the categories ASLEEP, off, on without dyskinesia, on with nontroublesome dyskinesia, and on with troublesome dyskinesia. See Hauser et al., J Clin Neuropharmacol 2000; 23:75-81.

A “Markov process” is a memoryless process satisfies the Markov property if one can make predictions for the future of the process based solely on its present state just as well as one could knowing the process's full history. i.e., conditional on the present state of the system, its future and past are independent.

The term “physiological measurements” is any wearable or non-contact measurement of body temperature with a clinical thermometer, or they may be more complicated, for example measuring how well the heart is functioning by taking an ECG (electrocardiograph.). Examples of transducers include:
Blood Pressure Cuff—measures systolic and diastolic blood pressure.
Force Plate—measures forces generated while stepping or jumping
Hand Dynamometer—strain-gage isometric dynamometer that measures grip strength
Nasal/Oral Thermocouple—monitors respiration through changes in temperature
Piezo-Electric Respiratory Effort Belt—measure respiration by recording chest or abdominal expansion and contraction.
Pulse Oximeter Finger Clip Sensor—measures blood oxygen saturation (SpO2) and pulse rate.
Spirometer—measures respiration, flow rate and lung volume.
GSR Sensor—measures galvanic skin response.
RIP Band—Respiratory Inductance Plethysmography band that monitors respiration and lung volume through chest expansion/contraction.
Push Button Event Marker—a push button held by subject or researcher to create event markers in software file
Skin Temperature Sensor—measure subject temperature on surface of the skin.

The term “physiological monitor” is the hardware and software combined in a sensor for measuring physiological measurements with any number of our sensors, and researchers are able to measure ECG, EEG, EMG, GSR, SpO2, respiration, force, and more:

A “policy” refers to rules and procedures setup for the subject of interest as it relates to treatment of an illness. The policy may be related to concentrations of drugs in body fluids, usually plasma, can be used during treatment and for diagnostic purposes. The selection of drugs for therapeutic drug monitoring is important. May times the concentrations of many drugs are not clearly related to their effects. For selected drugs therapeutic drug monitoring aims to enhance drug efficacy, reduce toxicity or assist with diagnosis.

A “subject of interest” refers to a human having a physiological function and being monitored for a drug's effectiveness. Although the term “human”, “person”, or “patient” may be used throughout this text, it should be appreciated that the subject may be something other than a human such as, for instance, an animal.

The term “vital signs” clinical measurements, specifically pulse rate, temperature, respiration rate, and blood pressure that indicates the state of essential body functions of a subject of interest's.

### Operating Environment

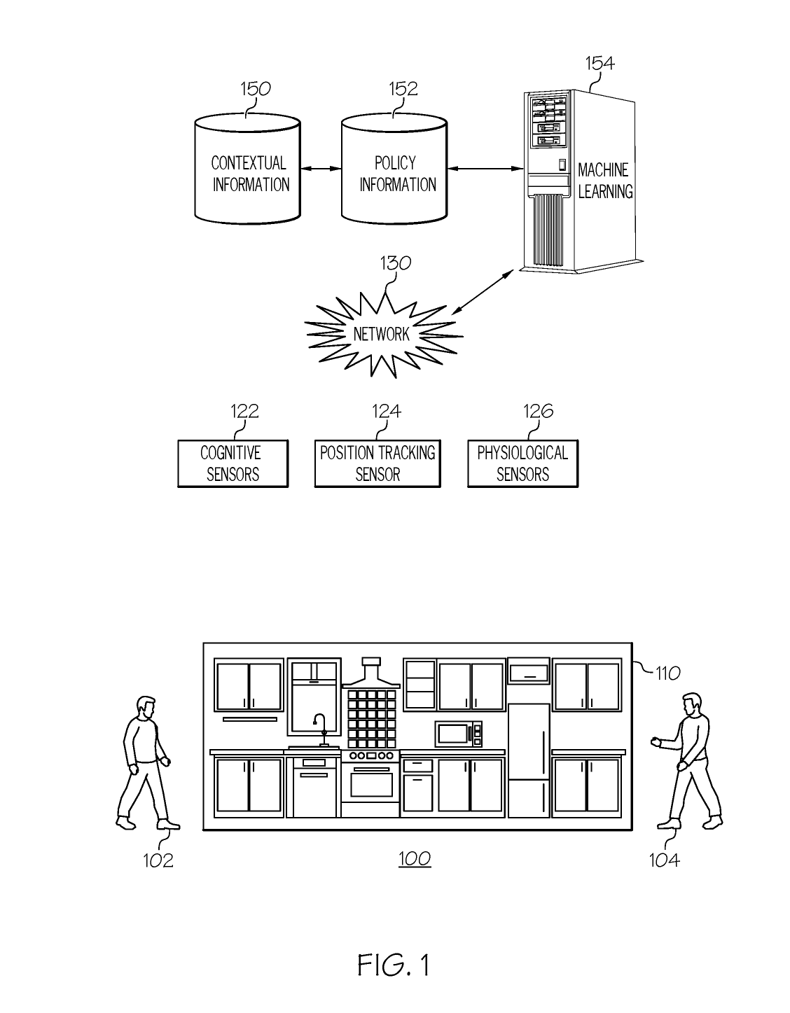

FIG. 1 illustrates one example of an operating environment 100 for managing a therapeutic state of a subject of interest. Shown is a kitchen area 110. The kitchen is just one example and any other physical venue is contemplated. A subject of interest 102 is walking into the kitchen area 110. The kitchen area 110 includes a variety of different sensors 122, 124, 126. The set of sensors broadly falls into three categories. A first set of cognitive based sensors 122 includes handheld devices. One example of a handheld device is a smartphone with a camera, a microphone, and a social media app. A second set of sensors to a derive location or position tracking 124 of the subject of interest 102 within the kitchen area 110. Location sensors may include a beacon or other low energy non-contact proximity sensor. A third set of sensors to derive vital signs and other physiological signals 126 of the subject of interest 102. Although these sets of sensors are described in three distinct sets, many of the technologies including cameras, location, microphones can be used for more than one type of sensing. For example, a camera can record a location as well as temperature in the infrared spectrum. Another example is a microphone can record speech patterns, the volume of speech or even respiratory function.

As shown the sensors 122, 124, 126 are connected to a network either wirelessly or wired back to computer system 154. The computer system 154 accesses information about the subject of interest including contextual information, i.e., does the subject of interest 102 typically go into the kitchen area at a given time. The computer system 154 also access information including policy information, e.g. the subject of interest 102 can only go into the kitchen if a care taker 104 is present.

### Stateless Transaction Manager

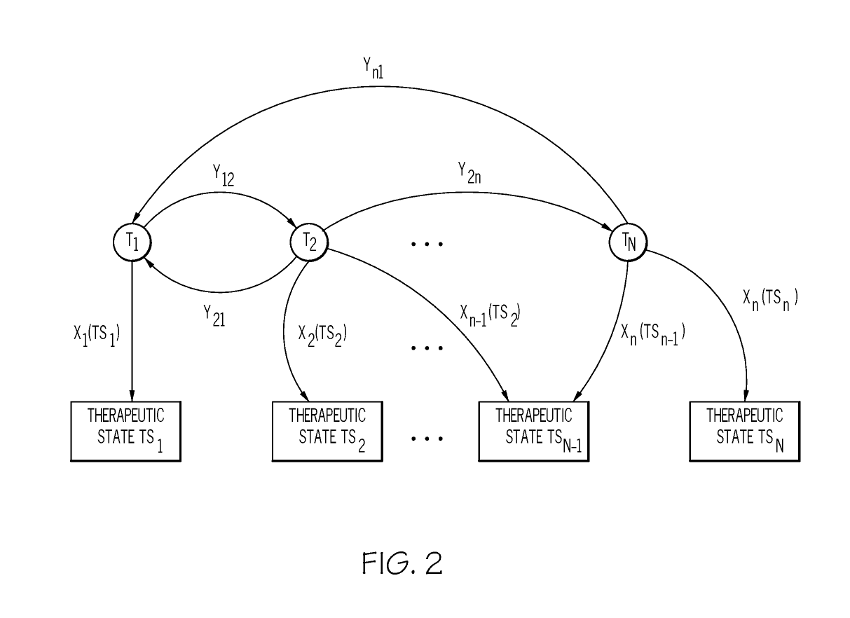

FIG. 2 is a view of a stateless transaction manager 128 that is part of the machine learning system of FIG. 1. One example is IBM Presence Insights software. However rather than using the IBM Presence Insights software for engaging customers with advertisement, it is set up, this is used to predict subject of interest's therapeutic state. Shown in this example is a simplified Markov process. The user moves from one therapeutic state (TS₁, TS₂ . . . TSₙ₋₁, TSₙ) with Transition (T₁, T₂ Tₙ₋₁, Tₙ) based on inputs (Y₁, Y₂ . . . Yₙ₋₁, Yₙ). The inputs are combination of contextual-based information on the subject of interest, cognitive-based information on the subject of interest, and policies or rules. As can be seen, the therapeutic state can change depending on this combination of inputs along with the current therapeutic state of the subject of interest is current.

More specifically, the machine learning algorithm predicts the therapeutic state. Contextual information such as location within a venue, time of day, dwell time at a location, i.e., how long the subject of interest remains performing an activity, such as brushing teeth, or preparing food or preparing food. The cognitive-based information can be derived from wearable technology, to determine heartrate, speech analytics, sleep patterns, posture, gait, gestural, and an accelerometer.

The machine learning algorithm relates these measurements and graphical representations to disease or drug therapeutic state that a user is in. That is a drug efficacy in stage III or IV trial. For example is “on” or “off' state in Parkinson disease state. This is typically entered in the Hauser diary.

Device movements are first filtered against location data and then checked against contextual and cognitive resources in order to determine an occurring action. The invention includes an inference engine in order to combine information from Location, Context, and Wearable Physiological Sensors to infer activities.

1. Location resources will allow the same movement types to be associated with different actions based on where the subject of interest is located within a venue. The location resources include:
Type of Room i.e., bedroom, kitchen, office, emergency room, stock room, etc.
Proximity to facility or device i.e., hospital monitor, refrigerator, computer workstation, TV, etc.
Dwell Times i.e., how long has this person been doing this action.
Location History i.e., how often is this person doing this action.
Coworker Data i.e., how many other individuals and which individuals are in the same location as the user.

2. Contextual resources might include:
Personal Information i.e., Is this person qualified to do this action? Is the nurse advised to perform this task on a patient? Should this person be cleaning the floors?
Weather i.e., should this person be walking on a surface that is not dry?
Time of Day i.e., should this person be locking up the doors at night? Should a person expect visitors at given time?
Social/News Alerts i.e., Is a real time event happening that would positively or negatively affect outcomes? Did a significant personal event just occur?

3. Cognitive Triggers might include:
Mobile Device Activity. Is this person wandering? Do they know what they are doing? Do they need help?
Biometrics. Is the user's heart rate relaxed and calm or is the user angry and upset. If the person has a high heart rate, maybe they are lifting something?
Online Speech Analysis. An ongoing analysis of speech is performed and inserted into the user's data stream.
Affective computing—much can be detected of cognitive state based on the users facial expression as one looks at a device with camera-fixed, laptop, mobile device, see (http://www.bostonmagazine.com/news/blog/2015/01/05/smiletracker-captures-photos-internet/).
Analysis of mobile device use—checking if the person is talking, text or browsing—content, direction and cadence of mobile use could detect cognitive state i.e., alert, distracted, hurried, bored, etc.
A “action” is then intelligently recommended to or noted for a person. This can be used for monitoring purposes and analytics can be performed.

### Example Use Cases

Hospital Setting: A nurse can recognize a condition of a patient.
Clinical Trial Setting: A clinician can recognize an outcome of a patient and how it relates to drug efficacy.
Remote patient/clinical trial monitoring: Data on patients are aggregated and analyzed to stratify patient cohorts and make determinations related to care, prescriptions, and clinical trial design and execution.

A nurse can recognize a condition of a patient tracks a subject of interest's movement combined with context information about patient's home, room, objects, particular spaces, to infer actions and possible actions that a patient typically engages in when these actions are consistent with a condition.

System may also compare patient's movement, i.e., the Markov process move from state to another state. Associated with these transitions, time of day, dwell time (i.e., how long brushing teeth), preparing food.

In clinical or hospital setting we relate these measurements and graphical representations of them to disease or drug therapeutic state that a user is in. In a drug efficacy in stage III or IV trial, for example, the states include “on” or “off' states in Parkinson disease, as in a Hauser diary.

A Markov process may also capture transitions between states.

“On State” describes whether drug is working, and may be correlated with whether a patient moves through a space, certain EMG measurements, certain rates of movement, and certain hear rates.

“Off state” describes when a drug is wearing off, or that a drug dose is wrong, or when a patient is just having a bad day. Different sets of patterns in the data may correlate with and describe these states.

Rather than querying the individual whether they are in ON or OFF state, the data may be queried instead.

Querying may then trigger an action that is recommended or noted. For example to record in the Hauser diary their state a helper notification may be sent to note state in diary. Such an alert helps remove burden from individual to remember, since they don't have to detect the transition in their state and remember to note it. Salience then is the measure, derived from a cognitive state that may or may not include subjective awareness.

The analysis of data and detection of a patient state may include a machine learning algorithm, which by training, appears to know what state the patient is in; they algorithms may also be trained to be predictive of an imminent state transition.

Based on these analytics and algorithms, a policy may be implemented in the system to track adverse reaction and to send an alert to a patient, nurse, caregiver, or clinician, thus providing a warning.

Such a warning may be to encourage a remote diagnosing in video conference.

### Managing A Therapeutic State Of A Subject of Interest Flow

The process starts in step 302 and immediately proceeds to step 304. In step 304, deriving location-based information from electronic sensors within a venue of a subject of interest using position tracking sensors 124. This location information includes how long it takes the subject of interest 102 to move to a specific spot within the venue 110. How long they stay or dwell at a spot, e.g., sink or stove. Who else 104 is near the subject of interest 102 in the venue 104.

Next in step 308, contextual-based information 150 on the subject of interest 102 is accessed. The contextual-based information includes personal information of the subject of interest, weather, time-of-day, educational background, media consumed, social network information, or a combination thereof. Cognitive-based information of the subject of interest 102 is derived from one or more cognitive or electronic sensors 122 in the venue 110 in step 306. The cognitive-base information includes mobile device activity, mobile data usage, biometrics, speech analysis, facial expressions, or a combination thereof.

A machine learning algorithm 154, in step 310, calculates using a combination of location-based information, contextual-based information, and cognitive-based information, to determine a therapeutic state of the subject of interest. The therapeutic state can be the current therapeutic state or a future therapeutic state.

Next in step 312, based on a predefined policy associated with the therapeutic state of the subject of interest, begin monitoring measurements of the subject of interest's vital signs in step 316. The vital signs in one example are additional vital signs not being previously monitored, e.g. sound of breathing. Or in another example, the vital signs are in addition to other vital signs not being monitored. In this example, the subject of interest may be directed to stand in front of a video monitor that begins a video-based telemedicine session. In this session other measurements such as infra-red may be used for analysis. Otherwise the process ends in step 318.

### Generalized Computing Environment

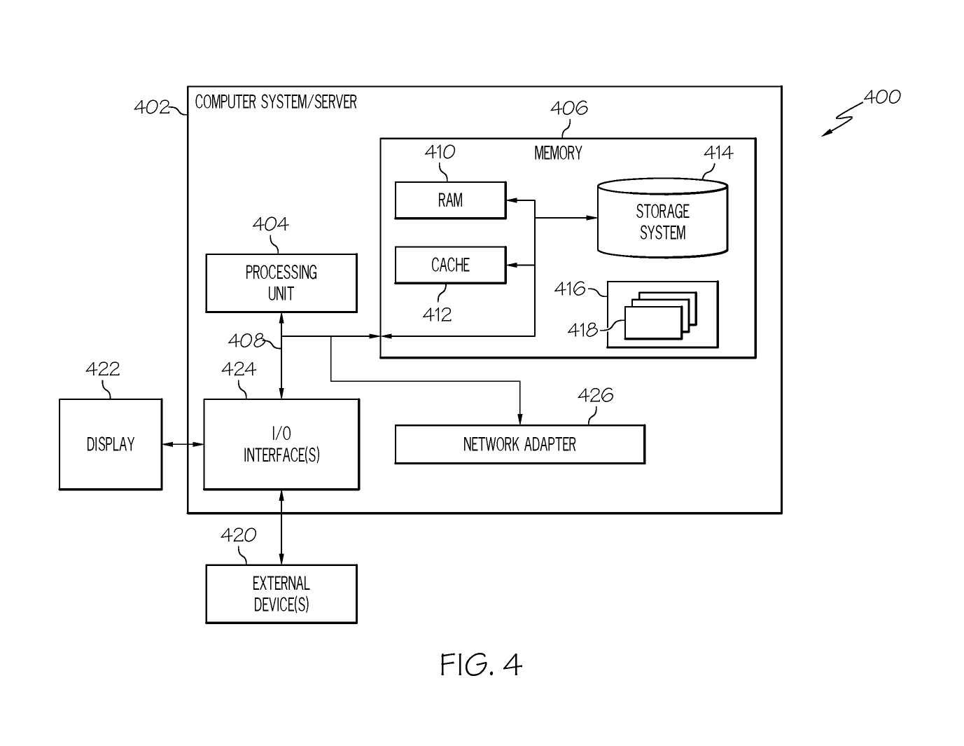

FIG. 4 illustrates one example of a processing node 400 for operating the stateless transaction manager 128 and is not intended to suggest any limitation as to the scope of use or functionality of embodiments of the invention described herein. Regardless, the computing node 400 is capable of being implemented and/or performing any of the functionality set forth hereinabove.

In computing node 400 there is a computer system/server 402, which is operational with numerous other general purpose or special purpose computing system environments or configurations. Examples of well-known computing systems, environments, and/or configurations that may be suitable for use with computer system/server 402 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 402 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 402 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 402 in cloud computing node 400 is shown in the form of a general-purpose computing device. The components of computer system/server 402 may include, but are not limited to, one or more processors or processing units 404, a system memory 406, and a bus 408 that couples various system components including system memory 406 to processor 404.

Bus 408 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 402 typically includes a variety of computer system readable media. Such media may be any available media that is accessible by computer system/server 402, and it includes both volatile and non-volatile media, removable and non-removable media.

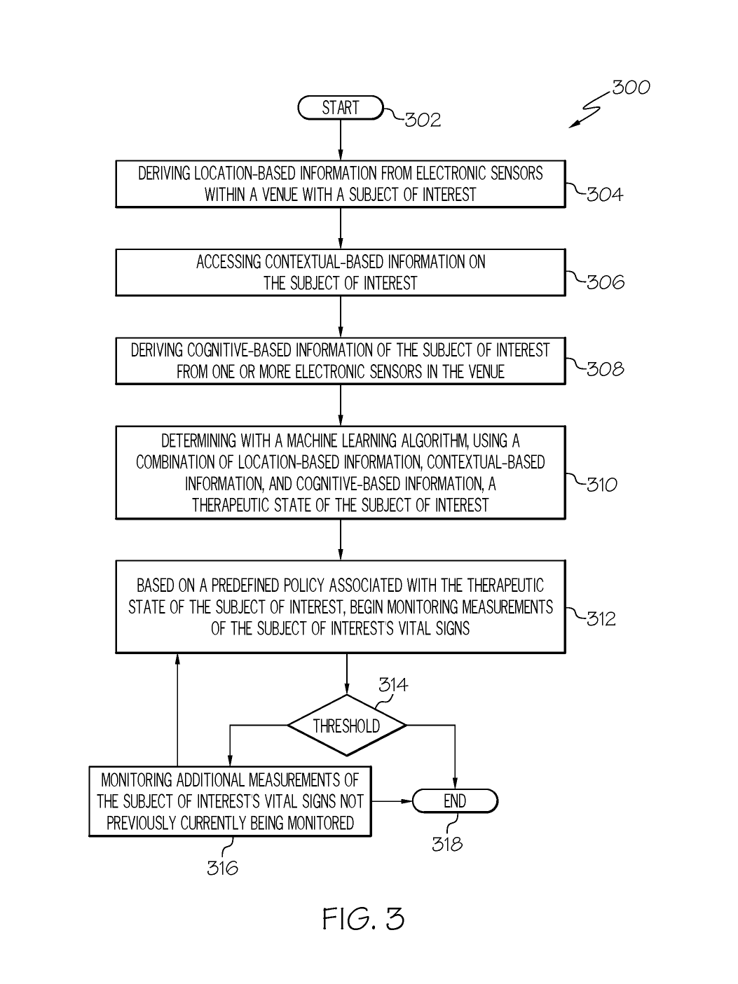

System memory 406, in one embodiment, implements the diagram of FIG. 1, FIG. 2 and the flow chart of FIG. 3. The system memory 406 can include computer system readable media in the form of volatile memory, such as random access memory (RAM) 410 and/or cache memory 412. Computer system/server 402 may further include other removable/non-removable, volatile/non-volatile computer system storage media. By way of example only, storage system 414 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 408 by one or more data media interfaces. As will be further depicted and described below, memory 406 may include at least one program product having a set (e.g., at least one) of program modules that are configured to carry out the functions of various embodiments of the invention.

Program/utility 416, having a set (at least one) of program modules 418, may be stored in memory 406 by way of example, and not limitation, as well as an operating system, one or more application programs, other program modules, and program data. Each of the operating system, one or more application programs, other program modules, and program data or some combination thereof, may include an implementation of a networking environment. Program modules 418 generally carry out the functions and/or methodologies of various embodiments of the invention as described herein.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon. The computer program product is typically non-transitory but in other examples it may be transitory.

Computer system/server 402 may also communicate with one or more external devices 1020 such as a keyboard, a pointing device, a display 422, etc.; one or more devices that enable a user to interact with computer system/server 402; and/or any devices (e.g., network card, modem, etc.) that enable computer system/server 402 to communicate with one or more other computing devices. Such communication can occur via I/O interfaces 424. Still yet, computer system/server 402 can communicate with one or more networks such as a local area network (LAN), a general wide area network (WAN), and/or a public network (e.g., the Internet) via network adapter 426. As depicted, network adapter 426 communicates with the other components of computer system/server 402 via bus 408. It should be understood that although not shown, other hardware and/or software components could be used in conjunction with computer system/server 402. Examples, include, but are not limited to: microcode, device drivers, redundant processing units, external disk drive arrays, RAID systems, tape drives, and data archival storage systems, etc.

### Cloud Computer Environment

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

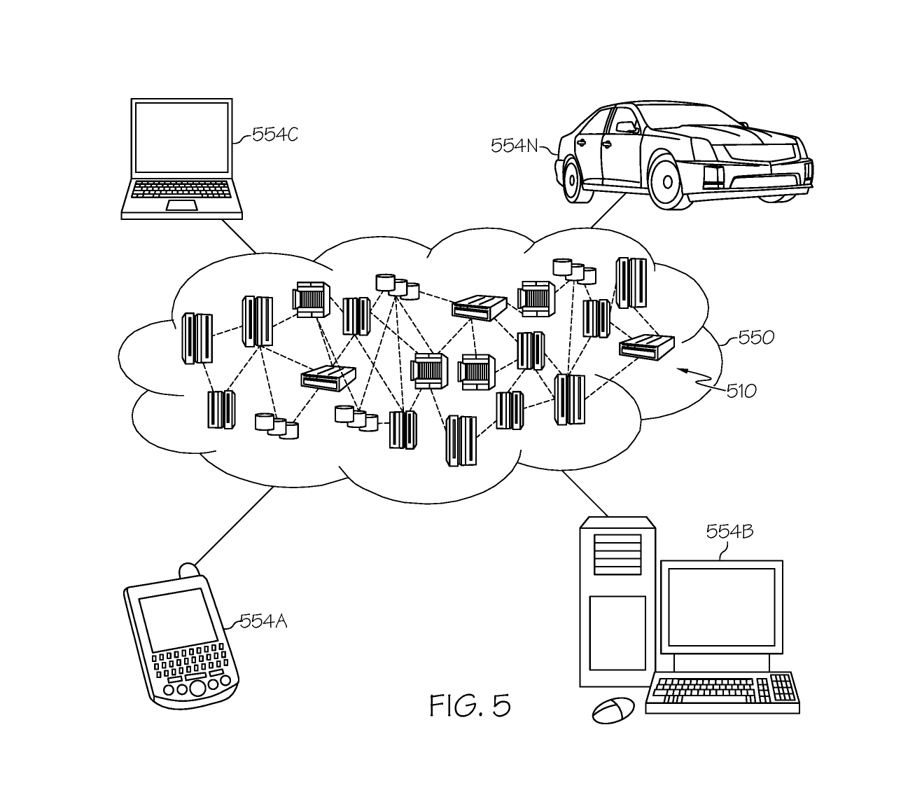

Referring now to FIG. 5, illustrative cloud computing environment 550 is depicted. As shown, cloud computing environment 550 comprises one or more cloud computing nodes 510 with which local computing devices used by cloud consumers, such as, for example, personal digital assistant (PDA) or cellular telephone 554A, desktop computer 554B, laptop computer 554C, and/or automobile computer system 554N may communicate. Nodes 510 may communicate with one another. They may be grouped (not shown) physically or virtually, in one or more networks, such as Private, Community, Public, or Hybrid clouds as described hereinabove, or a combination thereof. This allows cloud computing environment 550 to offer infrastructure, platforms and/or software as services for which a cloud consumer does not need to maintain resources on a local computing device. It is understood that the types of computing devices 554A-N shown in FIG. 5 are intended to be illustrative only and that computing nodes 510 and cloud computing environment 550 can communicate with any type of computerized device over any type of network and/or network addressable connection (e.g., using a web browser).

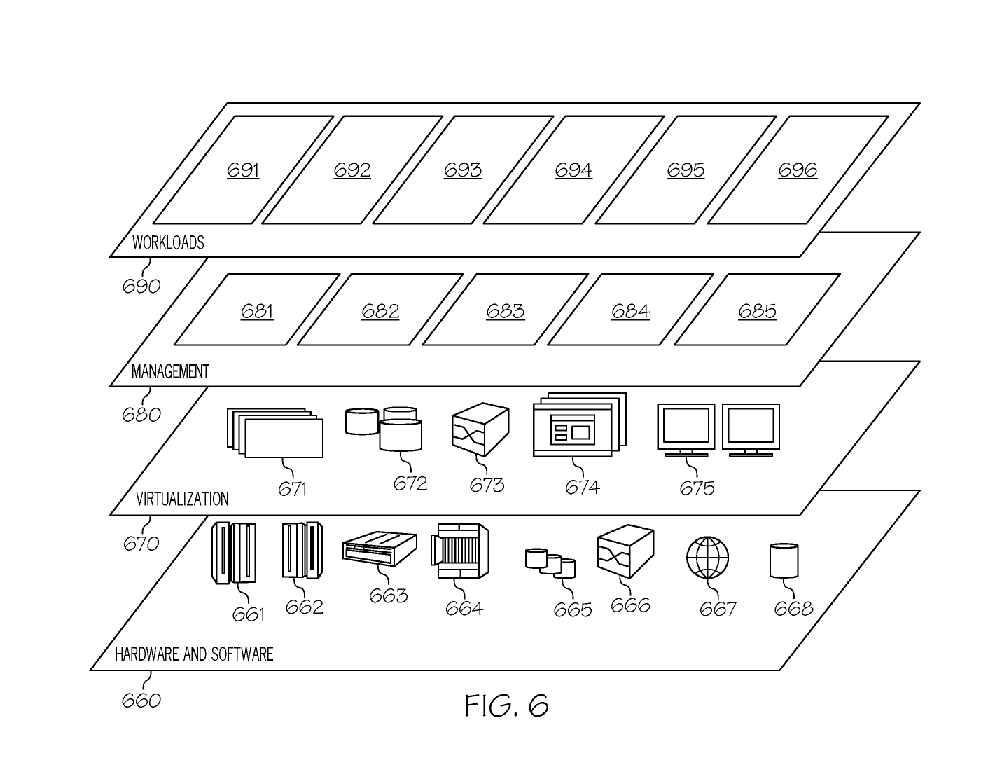

Referring now to FIG. 6, a set of functional abstraction layers provided by cloud computing environment 550 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 660 includes hardware and software components. Examples of hardware components include: mainframes 661; RISC (Reduced Instruction Set Computer) architecture based servers 662; servers 663; blade servers 664; storage devices 665; and networks and networking components 666. In some embodiments, software components include network application server software 667 and database software 668.

Virtualization layer 670 provides an abstraction layer from which the following examples of virtual entities may be provided: virtual servers 671; virtual storage 672; virtual networks 673, including virtual private networks; virtual applications and operating systems 674; and virtual clients 675.

In one example, management layer 680 may provide the functions described below. Resource provisioning 681 provides dynamic procurement of computing resources and other resources that are utilized to perform tasks within the cloud computing environment. Metering and Pricing 682 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 683 provides access to the cloud computing environment for consumers and system administrators. Service level management 684 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 690 provides examples of functionality for which the cloud computing environment may be utilized. Examples of workloads and functions which may be provided from this layer include: mapping and navigation 691; software development and lifecycle management 692; virtual classroom education delivery 693; data analytics processing 694; transaction processing 695; and for delivering services from a server to ensure multimedia content control by content providers (i.e., reduce piracy) and to ensure privacy by content users 696.

### Non-Limiting Examples

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method for managing a therapeutic state of a subject of interest, the computer-implemented method comprising:
deriving location-based information from electronic sensors within a venue of a subject of interest;
accessing contextual-based information on the subject of interest;
deriving cognitive-based information of the subject of interest from one or more electronic sensors in the venue;
determining, with a machine learning algorithm, using a combination of location-based information, contextual-based information, and cognitive-based information, a therapeutic state of the subject of interest; and
based on a predefined policy associated with the therapeutic state of the subject of interest, begin monitoring measurements of the subject of interest's vital signs.

2. The computer-implemented of claim 1, further comprising:
deriving measurements of the subject of interest vital signs from electronic sensors within the venue; and wherein based on a predefined policy associated with the therapeutic state of the subject of interest, begin monitoring additional measurements of the subject of interest's vital signs not previously currently being monitored.

3. The computer-implemented of claim 1, wherein the deriving location-based information from electronic sensors within a venue with a subject of interest includes at least one of dwell times at a particular location within the venue and other subjects within the venue.

4. The computer-implemented of claim 1, wherein the accessing contextual-based information on the subject of interest includes at least one of personal information of the subject of interest, weather, time-of-day, educational background, media consumed, and social network information.

5. The computer-implemented of claim 1, wherein the deriving cognitive-based information of the subject of interest from one or more electronic sensors in the venue includes at least one of the subject of interest's mobile device activity, mobile data usage, biometrics, speech analysis, posture, gait, gestural, and facial expressions.

6. The computer-implemented of claim 1, wherein the monitoring measurements of the subject of interest's vital signs includes initiating a video-based telemedicine session.

7. The computer-implemented of claim 1, wherein the therapeutic state of the subject of interest includes a future therapeutic state of the subject of interest.

8. The computer-implemented of claim 1, wherein the therapeutic state of the subject of interest is one of an “ON” state or and “OFF” state used with a Parkinson disease state; and further comprising:
making an entry in a journal of the therapeutic state along with a time and date of the entry.

9. The computer-implemented of claim 1, wherein the electronic sensors include one or more of wearable devices, physiological monitors, cameras, accelerometers, and microphones.

10. A system for managing a therapeutic state of a subject of interest, the system comprising:
a memory;
a processor communicatively coupled to the memory, where the processor is configured to perform
deriving location-based information from electronic sensors within a venue of a subject of interest;
accessing contextual-based information on the subject of interest;
deriving cognitive-based information of the subject of interest from one or more electronic sensors in the venue;
determining, with a machine learning algorithm, using a combination of location-based information, contextual-based information, and cognitive-based information, a therapeutic state of the subject of interest; and
based on a predefined policy associated with the therapeutic state of the subject of interest, begin monitoring measurements of the subject of interest's vital signs.

11. The system of claim 10, further comprising:
deriving measurements of the subject of interest vital signs from electronic sensors within the venue; and wherein based on a predefined policy associated with the therapeutic state of the subject of interest, begin monitoring additional measurements of the subject of interest's vital signs not previously currently being monitored.

12. The system of claim 10, wherein the deriving location-based information from electronic sensors within a venue with a subject of interest includes at least one of dwell times at a particular location within the venue and other subjects within the venue.

13. The system of claim 10, wherein the accessing contextual-based information on the subject of interest includes at least one of personal information of the subject of interest, weather, time-of-day, educational background, media consumed, and social network information.

14. The system of claim 10, wherein the deriving cognitive-based information of the subject of interest from one or more electronic sensors in the venue includes at least one of the subject of interest's mobile device activity, mobile data usage, biometrics, speech analysis, posture, gait, gestural, and facial expressions.

15. The system of claim 10, wherein the monitoring measurements of the subject of interest's vital signs includes initiating a video-based telemedicine session.

16. The system of claim 10, wherein the therapeutic state of the subject of interest includes a future therapeutic state of the subject of interest.

17. The system of claim 10, further comprising:
deriving measurements of the subject of interest vital signs from electronic sensors within the venue; and wherein based on a predefined policy associated with the therapeutic state of the subject of interest, begin monitoring additional measurements of the subject of interest's vital signs not previously currently being monitored.

18. The system of claim 10, wherein the deriving location-based information from electronic sensors within a venue with a subject of interest includes at least one of dwell times at a particular location within the venue and other subjects within the venue.

19. A non-transitory computer program product for managing a therapeutic state of a subject of interest comprising a computer readable storage medium having computer readable program code embodied therewith, the computer readable program code configured to perform:
deriving location-based information from electronic sensors within a venue of a subject of interest;
accessing contextual-based information on the subject of interest;
deriving cognitive-based information of the subject of interest from one or more electronic sensors in the venue;
determining, with a machine learning algorithm, using a combination of location-based information, contextual-based information, and cognitive-based information, a therapeutic state of the subject of interest; and
based on a predefined policy associated with the therapeutic state of the subject of interest, begin monitoring measurements of the subject of interest's vital signs.

20. The non-transitory computer program product of claim 19, further comprising:
deriving measurements of the subject of interest vital signs from electronic sensors within the venue; and wherein based on a predefined policy associated with the therapeutic state of the subject of interest, begin monitoring additional measurements of the subject of interest's vital signs not previously currently being monitored.
